(19) United States
(12) Patent Application Publication
(10) Pub. No.: US 2014/0052196 A1
(43) Pub. Date: Feb. 20, 2014

(54) HAMMER TOE IMPLANT WITH LIVING HINGE AND METHOD

(71) Applicant: Wright Medical Technology, Inc., Arlington, TX (US)

(72) Inventors: Shawn E. McGinley, Arlington, TN (US); Mary J. McCombs-Stearnes, Lakeland, TN (US)

(73) Assignee: Wright Medical Technology, Inc., Arlington, TN (US)

(21) Appl. No.: 14/067,096

(22) Filed: Oct. 30, 2013

Related U.S. Application Data
(63) Continuation-in-part of application No. 13/086,136, filed on Apr. 13, 2011.

(51) Int. Cl. A61B 17/86

(52) U.S. Cl. CPC A61B 17/8605
USPC 606/319

(57) ABSTRACT

An implant is disclosed including an elongate threaded portion and a blade extending from the elongate threaded portion. The blade having a taper terminating at a point and a biasable cantilever formed in a central portion of said blade and with a free end that protrudes from at least one surfaces when the cantilever is unbiased.

### CROSS-REFERENCE TO RELATED APPLICATIONS

This application is a Continuation-in-Part of pending patent application Ser. No. 13/086,136, filed Apr. 13, 2011, which claims priority to U.S. Provisional Patent Application Ser. No. 61/350,665, filed on Jun. 2, 2010, the entirety of which are herein incorporated by reference.

### FIELD OF DISCLOSURE

The disclosed system and method relate implants. More specifically, the disclosed system and method relate to installing an implant for treating hammer toe.

### BACKGROUND

Hammer toe is a deformity of the toe that affects the alignment of the bones adjacent to the proximal interphalangeal (PIP) joint. Hammer toe can cause pain and can lead to difficulty in walking or wearing shoes. A hammer toe can often result in an open sore or wound on the foot. In some instances, surgery may be required to correct the deformity by fusing one or both of the PIP and distal interphalangeal (DIP) joints.

The most common corrective surgery includes the placement of a pin or rod in the distal, middle, and proximal phalanxes of the foot to fuse the PIP and DIP joints. The pin or rod is cut at the tip of the toe, externally of the body. A plastic or polymeric ball is placed over the exposed end of the rod, which remains in the foot of the patient until the PIP and/or DIP joints are fused in approximately 6 to 12 weeks. This conventional treatment has several drawbacks such as preventing the patient from wearing closed toe shoes while the rod or pin is in place, and the plastic or polymeric ball may snag a bed sheet or other object due to it extending from the tip of the toe resulting in substantial pain for the patient.

Another conventional implant includes a pair of threaded members that are disposed within adjacent bones of a patient's foot. The implants are then coupled to one another through male-female connection mechanism, which is difficult to install in situ and has a tendency to separate.

Yet another conventional implant has body including an oval head and a pair of feet, which are initially compressed. The implant is formed from nitinol and is refrigerated until it is ready to be installed. The head and feet of the implant expand due to the rising temperature of the implant to provide an outward force on the surrounding bone when installed. However, the temperature sensitive material may result in the implant deploying or expanding prior to being installed, which requires a new implant to be used.

Accordingly, an improved implant for treating hammer toe is desirable.

### SUMMARY

An implant is disclosed including an elongate threaded portion and a blade extending from the elongate threaded portion. The blade has a taper terminating at a point and a biasable cantilever formed in a central portion and having a free end that protrudes from at least one surface when said cantilever is unbiased.

A method is also disclosed in which an incision is formed to gain access to a joint between first and second bones. The first and second bones are flexed such that the bones are disposed at an angle from one another. A threaded portion of an implant is advanced into the first bone. The implant includes a blade portion extending from the elongate threaded portion. The second bone is repositioned such that a middle of the second bone is approximately aligned with the blade portion of the implant. The second bone is forced into engagement with the blade portion of the implant.

A surgical assembly is disclosed comprising an implant having an elongate body and a driving assembly. The implant includes a threaded end and a blade end extending from the threaded end. The blade end tapers along its thickness and its width to a point and includes a plurality of serrated edges and a biasable cantilever formed in a central portion and having a free end that protrudes from at least one surface when said cantilever is unbiased. The driving assembly includes a handle, a driving rod extending from the handle, and an adapter coupled to an end of the driving rod. The adapter has a body defining a slot at one end that is sized and configured to receive the blade end of the implant.

### BRIEF DESCRIPTION OF THE DRAWINGS

These and other features and advantages of the present invention will be more fully disclosed in, or rendered obvious by the following detailed description of the preferred embodiments of the invention, which are to be considered together with the accompanying drawings wherein like numbers refer to like parts and further wherein:

FIG. 1 is an isometric view of one example of an improved hammer toe implant;

FIG. 2 is a side view of the hammer toe implant illustrated in FIG. 1;

FIG. 3 is a sectional view of the hammer toe implant taken along line 3-3 in FIG. 2;

FIG. 3A is an isometric view of another example of an improved hammer toe implant;

FIG. 3B is a plan view of the improved hammer toe implant shown in FIG. 3A;

FIG. 3C is a side view of the improved hammer toe implant shown in FIG. 3A;

FIG. 3D is an isometric view of a further example of improved hammer toe implant according to the invention;

FIG. 3E is a plan view of the improved hammer toe implant shown in FIG. 3A;

FIG. 3F is a side view of the improved hammer toe implant shown in FIG. 3A;

FIG. 4 is an end on view of the hammer toe implant taken along line 4-4 in FIG. 2;

FIG. 5 is a top view of another example of a hammer toe implant;

FIG. 6 is a side view of the hammer toe implant illustrated in FIG. 5;

FIG. 7 is a side view of one example of a driving adapter for use with the hammer toe implants illustrated in FIGS. 1 and 6;

FIG. 8 is an end view of the driving adapter illustrated in FIG. 7;

FIG. 9 is a side view of another example of a driving adapter for use with the hammer toe implants illustrated in FIGS. 1 and 6;

FIG. 10 is an end view of the driving adapter illustrated in FIG. 9;

FIG. 11 is an assembly view of a hammer toe implant engaged by a driving adapter;

FIGS. 12A and 12B illustrate the middle and proximal phalanxes of a foot being resected;

FIG. 13 illustrates a hammer toe implant being driven into a proximal phalanx;

FIG. 14 illustrates a middle phalanx being drilled or broached;

FIG. 15 illustrates a blade of a hammer toe implant extending from the proximal phalanx with the middle phalanx having been drilled or broached;

FIG. 16 illustrates a hammer toe implant installed in the middle and proximal phalanxes;

FIG. 17 illustrates another example of a driving assembly for installing an implant;

FIG. 18 illustrates side view of the driving assembly illustrated in FIG. 17;

FIG. 19 is an isometric view of an adapter of the driving assembly illustrated in FIG. 17;

FIG. 20 is an end view of the adapter illustrated in FIG. 19;

FIG. 21 is a cross-sectional view of the adapter taken along line 21-21 in FIG. 20;

FIG. 22 is a cross-sectional view of the adapter taken along line 22-22 in FIG. 20;

FIG. 23 is a plan view of the driving rod of the driving assembly illustrated in FIG. 17;

FIG. 24 is a cross-sectional view of the driving rod taken along line 24-24 in FIG. 23;

FIG. 25 is a cross-sectional view of the fin of the driving rod taken along line 25-25 in FIG. 23;

FIG. 26 is a plan view of driving assembly illustrated in FIG. 17 without the o-ring;

FIG. 27 is a cross-sectional view of the handle taken along line 27-27 in FIG. 26;

FIGS. 28A and 28B illustrate the middle and proximal phalanxes of a foot being resected;

FIGS. 29A and 29B illustrate an implant coupled to the adapter of the driving assembly illustrated in FIG. 17;

FIG. 30 illustrates a hammer toe implant being driven into a proximal phalanx;

FIG. 31 illustrates a middle phalanx being drilled or broached;

FIG. 32 illustrates a blade of a hammer toe implant extending from the proximal phalanx with the middle phalanx having been drilled or broached; and

FIG. 33 illustrates a hammer toe implant installed in the middle and proximal phalanxes.

### DETAILED DESCRIPTION

This description of preferred embodiments is intended to be read in connection with the accompanying drawings, which are to be considered part of the entire written description. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral,” and the like are to be interpreted relative to one another or relative to an axis of elongation, or an axis or center of rotation, as appropriate. Terms concerning attachments, coupling, and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship.

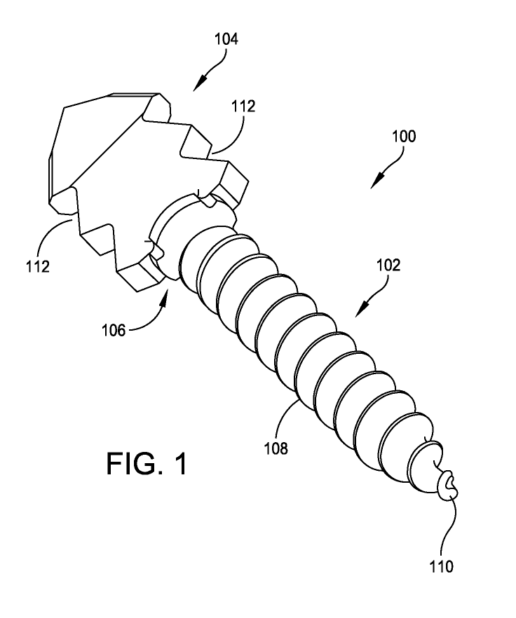

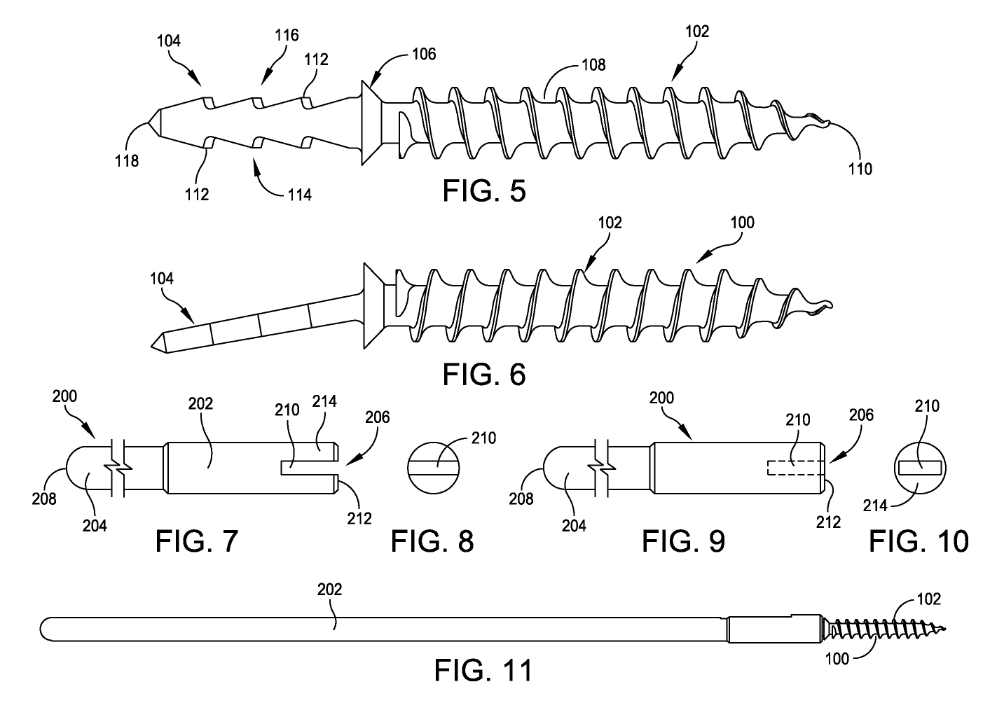

FIG. 1 illustrates one example of an implant 100 for treating hammer toe. As shown in FIG. 1, implant 100 includes a threaded portion 102 and a blade portion 104, which are connected together at an engagement portion 106. Implant 100 may have a substantially linear geometry having an overall length of approximately 19 mm (approximately 0.75 inches). In some embodiments, such as the one illustrated in FIGS. 5 and 6, blade portion 104 may be disposed at angle with respect to a longitudinal axis defined by the threaded portion 102. The angle may be between zero and 45 degrees, and more particularly between approximately five and fifteen degrees, although one skilled in the art will understand that implant 100 may have other dimensions and be provided in different sizes. For example, implant 100 may be provided in lengths of 16 mm and 22 mm, to name a few potential lengths.

Threaded portion 102 may include a plurality of threads 108 disposed along its entire length, which may be approximately 13 mm (approximately 0.5 inches). The tip 110 of threaded portion 102 may be pointed to facilitate the advancement of threads 108 into bone. Threads 108 may have a maximum outer diameter of approximately 2 mm (approximately 0.08 inches), although one skilled in the art will understand that thread portion 102 may have other dimensions and be configured to be received within a phalanx bone of a person. For example, threads may have an outer diameter of approximately 2.4 mm and 1.6 mm, to name a few potential possibilities.

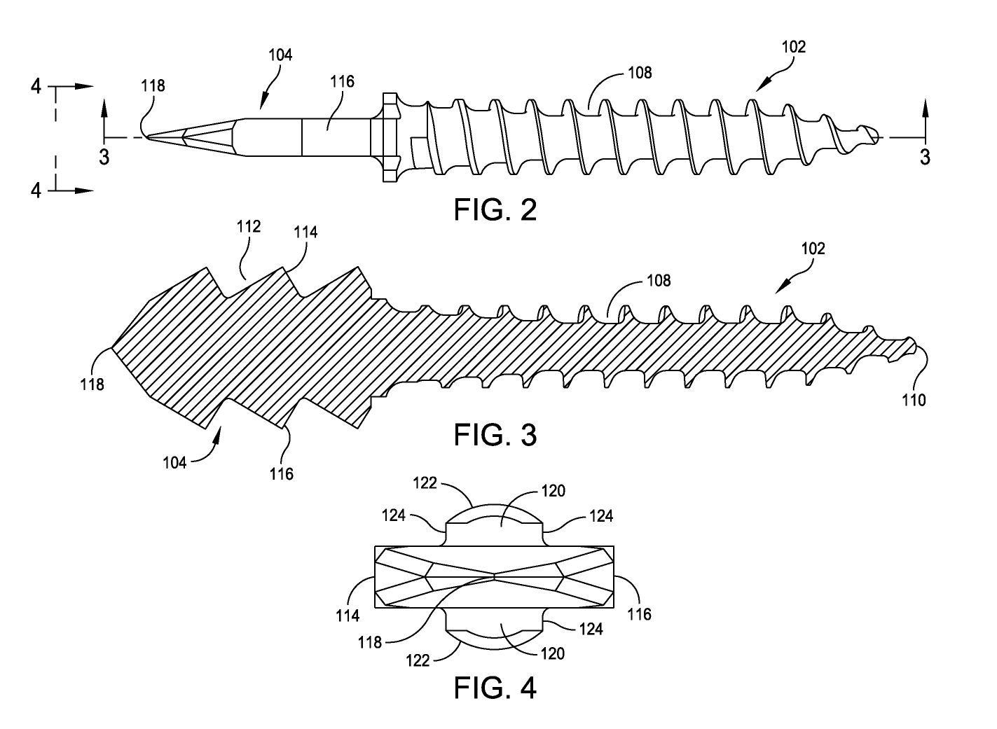

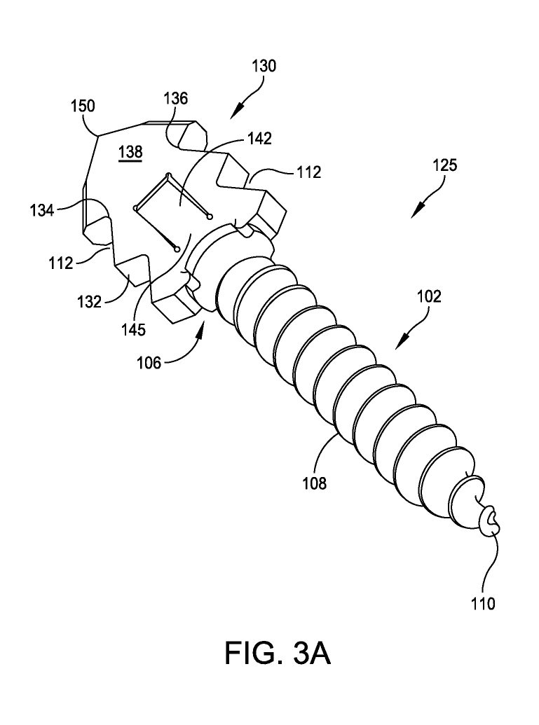

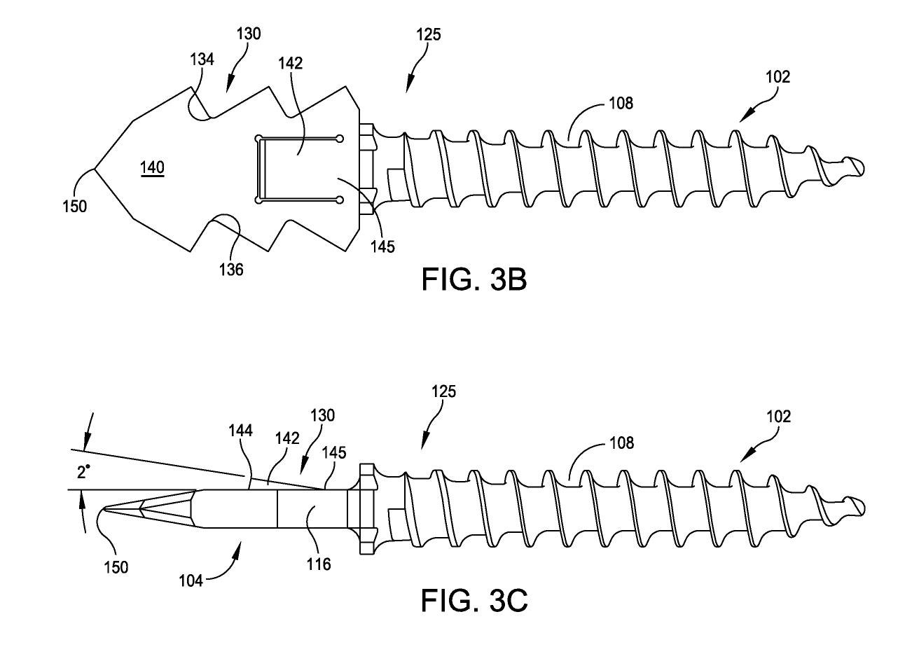

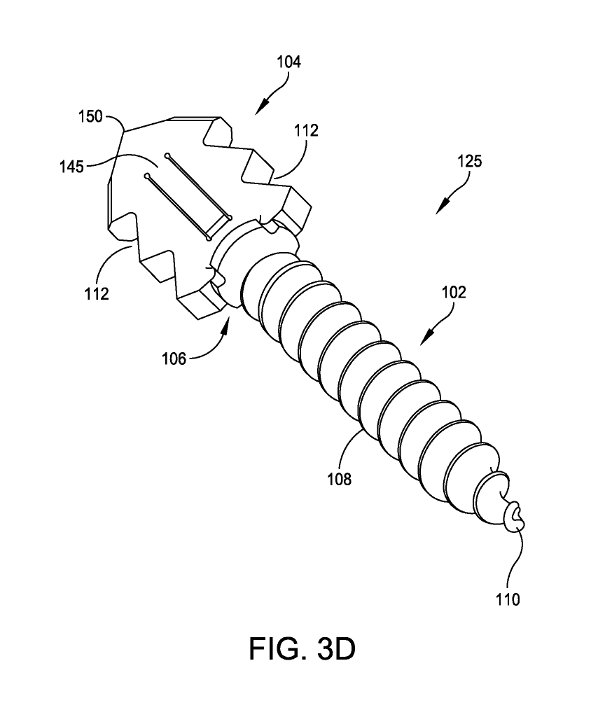

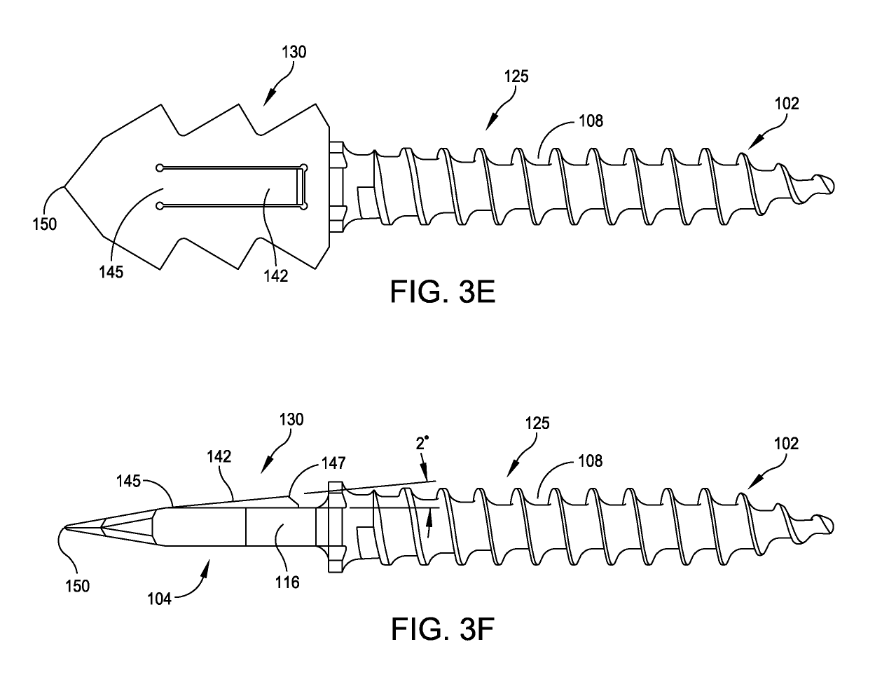

As best seen in FIG. 3, blade portion 104 includes a plurality of serrated edges 112 on its top and bottom sides 114, 116. Blade portion 104 may have a width that is greater than its thickness as best seen in FIGS. 2 and 4. For example, blade portion 104 may have a width of approximately 0.4 centimeters (approximately 0.16 inches) and a thickness of approximately 0.1 centimeters (approximately 0.04 inches) each of which taper to point 118. Blade portion 104 may have a substantially rectangular cross-sectional area as illustrated in FIG. 4, although one skilled in the art will understand that blade portion 104 may have other cross-sectional geometries. Engagement portion 106 may include a pair of protrusions 120 extending from opposite sides of implant 100 and having rounded outer edges 122. The sides 124 of protrusions 120 may be substantially parallel with each other as shown in FIG. 4.

Referring to FIGS. 3A-3F, one embodiment of the invention provides an implant 125 that includes an alternative blade portion 130 that includes a plurality of serrations 132 formed along edges 134, 136, a top surface 138, and a bottom surface 140. Blade portion 130 often has a width that is greater than its thickness. A cantilever 142 is formed in blade portion 130 between edges 134, 136. Cantilever 142 includes a free end and a clamped end 145 that is located in spaced relation to the free end. The free end of cantilever 142 is often formed so as to have a chamfer 147 (FIG. 3F) along a top edge. In some embodiments, clamped end 145 is located within blade portion 130 adjacent to engagement portion 106 (FIGS. 3A-3B). In these embodiments, the free end of cantilever 142 is located in spaced relation to engagement portion 106. In other embodiments, clamped end 145 is located within blade portion 130 in spaced relation to engagement portion 106, but with free end 147 being located adjacent to engagement portion 106 (FIGS. 3D-3F). In most embodiments, cantilever 142 will be pre-loaded such that chamfered free end 147 will stand proud of either top surface 138 or bottom surface 140 often by a distance approximating the thickness of blade portion 130. In this arrangement, cantilever 142 is inclined at about a 2° angle relative to top surface 138 or bottom surface 140. Often, chamfered free end 147 of cantilever 142 will rise above top surface 138 so that cantilever 142 will form an angle of approximately 2°-4° with respect to top surface 138. For example, blade portion 130 may have a width of approximately 0.4 centimeters (approximately 0.16 inches) and a thickness of approximately 0.1 centimeters (approximately 0.04 inches) each of which taper to point 150. Blade portion 130 may have a substantially rectangular cross-sectional area, although one skilled in the art will understand that blade portion 130 may have other cross-sectional geometries. For example, in some embodiments, blade portion 130 may taper along its width and thickness to point 150.

Implants 100 and 125 are both configured to be installed using a driving adapter 200 such as the one illustrated in FIGS. 7-10. The driving adapter 200 has an elongate body 202 having a proximal end 204 and a distal end 206. Body 202 of driving adapter 200 may have a circular cross-sectional geometry, although one skilled in the art will understand that body 202 may have other cross-sectional geometries including, but not limited to, triangular, rectangular, pentagonal, and hexagonal to name a few.

Proximal end 204 may be substantially solid and have a rounded tip 208. Distal end 206 may define a slot 210 sized and configured to receive blade portion 104,130 of implants 100, 125, respectively. Slot 210 may have a rectangular cross-sectional geometry and have a depth that is sufficient to receive the entire blade portion 104 of implant 100 such that distal edges 212 of slot 210 contact protrusions 120 of engagement portion 106. However, one skilled in the art will understand that slot 210 may have other cross-sectional geometries and dimensions. Slot 210 may extend through side walls 214 of body 202 as shown in FIGS. 7 and 8, or side walls 214 may completely enclose slot 210 as shown in FIGS. 9 and 10.

If the driving adapter 200 is to be used with an implant 100 having a substantially linear lengthwise geometry such as the implants 100, 125 illustrated in FIGS. 1-5, then slot 210 may extend in a direction that is substantially parallel to an axis defined by body 202 of driving adapter 200. If driving adapter 200 is to be used with an implant 100 having a blade portion 104 that extends at an angle with respect to an axis defined by elongate threaded portion 102 such as the implant illustrated in FIGS. 5 and 6, then slot 210 may extend from distal edges 212 at an angle with respect to an axis defined by the length of body 202 such that elongate threaded portion 102 of implant 100 is linearly aligned with body 202 of driving adapter 200 as shown in FIG. 11. For example, if blade portion 104 of implant 100 extends at a ten degree angle with respect to an axis defined by elongate threaded portion 102, then slot 210 of driving adapter 200 may extend at a ten degree angle with respect to a longitudinal axis defined by body 202 such that threaded portion 102 of implant 100 and body 202 of driving adapter 200 are substantially linearly aligned.

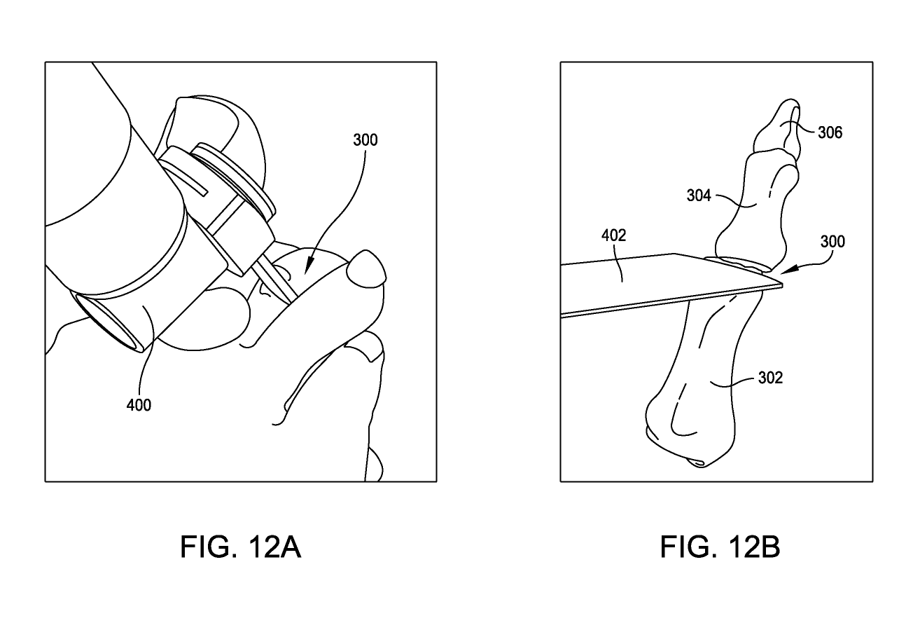

A method for installing implants 100, 125 in the proximal interphelangeal joint (PIP) 300 is described with reference to FIGS. 12A-16. However, one skilled in the art will understand that the technique for installing implants 100, 125 may be applied to other joints such as, for example, the distal interphelangeal (DIP) joint between middle phalanx 304 and distal phalanx 306. As shown in FIGS. 12A and 12B, an incision is made to open the PIP joint 300 and a cutting tool 400 having a blade 402 may be used to resect adjacent faces of proximal phalanx 302 and middle phalanx 304. The resected surfaces of proximal phalanx 302 and middle phalanx 304 may be debrided as understood by one skilled in the art.

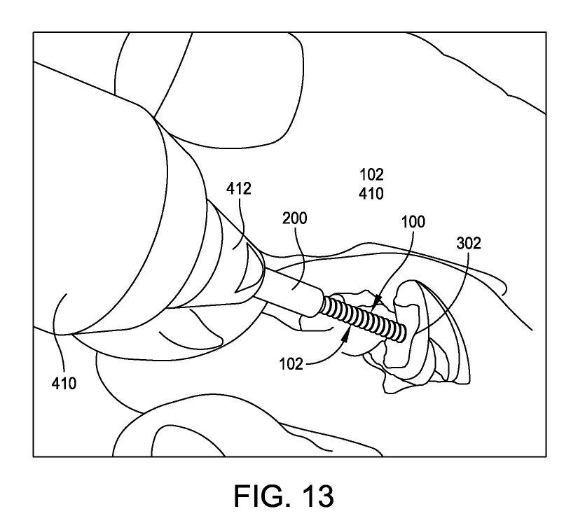

Blade portions 104, 130 of implants 100, 125 may be disposed within slot 210 of driving adapter 200 as shown in FIG. 11, and the body 202 of driving adapter 200 may be secured in a chuck 412 of a drill 410 or other driving instrument as shown in FIG. 13. Drill 410 or other driving instrument is used to drive the threaded portion 102 of implants 100, 125 into the resected surface of proximal phalanx 302. With the threaded portion 104, 130 of implants 100, 125 disposed within proximal phalanx 302, driving adapter 200 may be disengaged from blade portion 104, 130 of implants 100, 125.

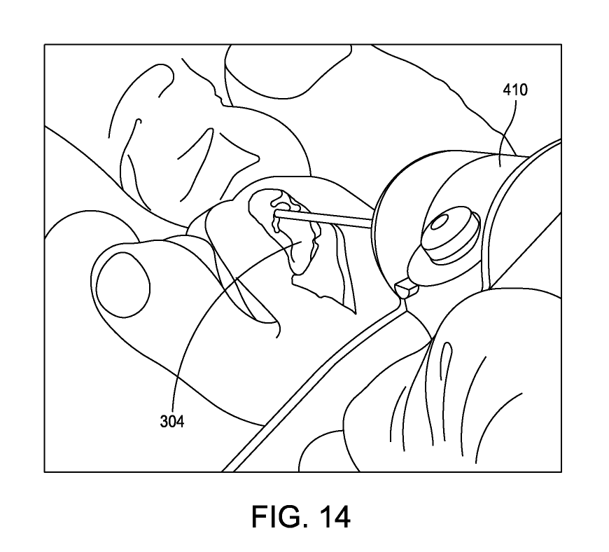

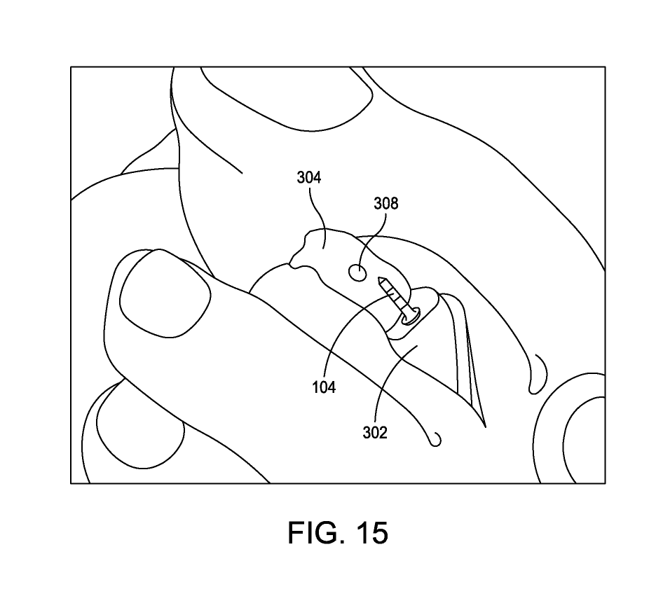

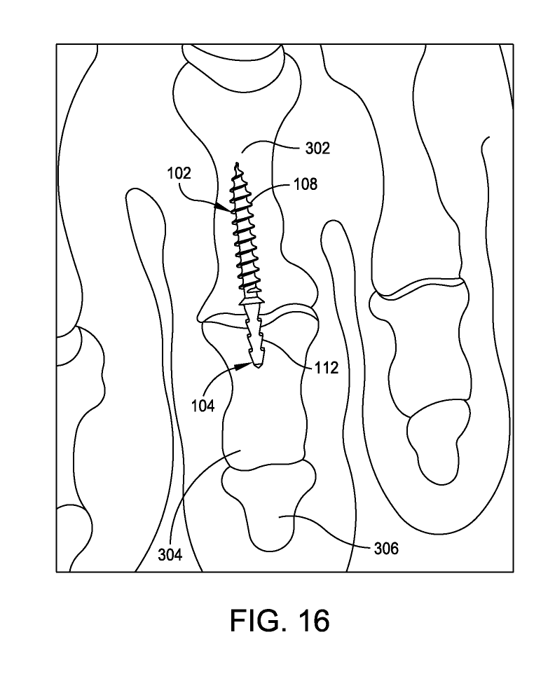

Middle phalanx 304 may be predrilled or broached using drill 410 to create a hole 308 as shown in FIGS. 14 and 15. The predrilled or broached middle phalanx 304 is then repositioned such that the predrilled hole or broach 308 aligns with the blade portion 104 of implant 100. The middle phalanx 304 is then pressed into engagement with the blade portion 104 as shown in FIG. 16. Serrated edges 112 of blade portion 104 help to maintain the engagement between middle phalanx 304 and blade portion 104 of implant 100.

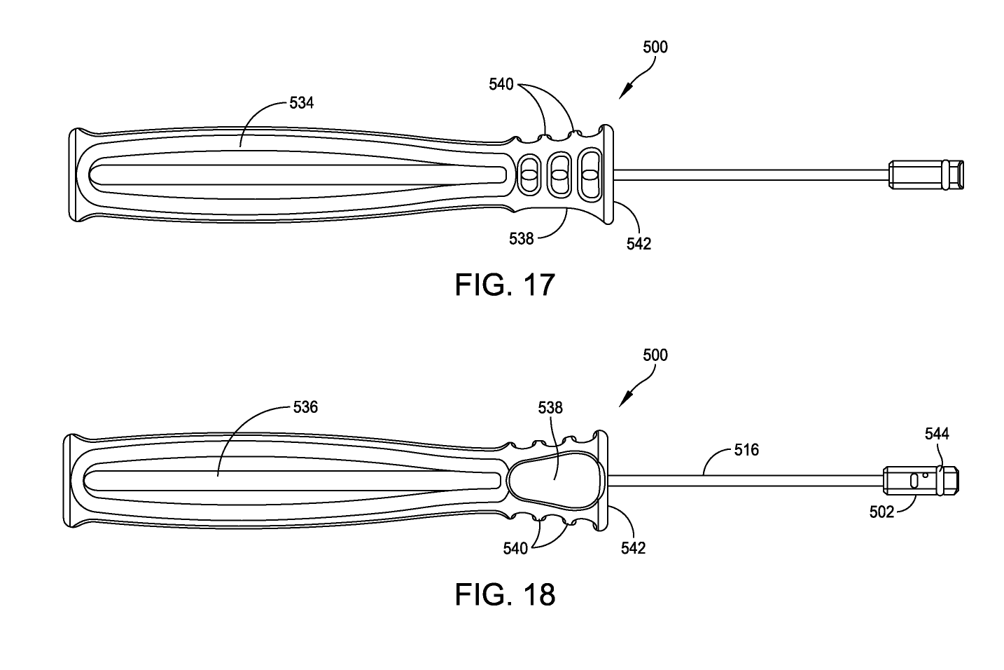

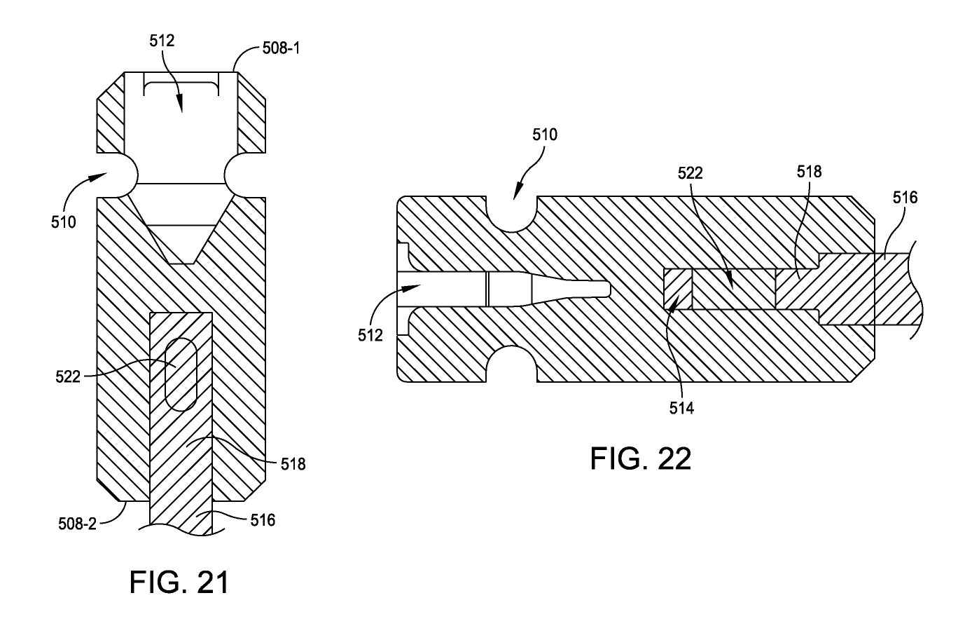

A further method for installing implant 125 in the proximal interphelangeal joint (PIP) 300 is substantially similar to the method used in connection with implant 100 and illustrated in FIGS. 12A-16 and 28A-33, utilizing tooling as shown in FIGS. 17-27. More particularly, an incision is made to open the PIP joint 300 with a cutting tool 400 having a blade 402 used to resect adjacent faces of proximal phalanx 302 and middle phalanx 304. Blade portion 130 of implant 125 may also be disposed within slot 210 of driving adapter 200, and the body 202 of driving adapter 200 may be secured in a chuck 412 of a drill 410 or other driving instrument. Drill 410 or other driving instrument is used to drive the threaded portion 102 of implant 125 into the resected surface of proximal phalanx 302. With the threaded portion 102 of implant 125 disposed within proximal phalanx 302, driving adapter 200 may be disengaged from blade portions 104, 130 of implants 100, 125. Middle phalanx 304 may be predrilled or broached using drill 410 to create a hole 308. The predrilled or broached middle phalanx 304 is then repositioned such that the predrilled hole or broach 308 aligns with the blade portions 104, 130 of implants 100, 125. The middle phalanx 304 is then pressed into engagement with the blade portion 130. Serrated edges 112 of blade portion 130 help to maintain the engagement between middle phalanx 304 and blade portion 130 of implant 125. Advantageously, as blade portion 130 enters predrilled hole or broach 308 of middle phalanx 304 cantilever 142 is biased toward blade portion 130 by the edge of middle phalanx 304 that defines predrilled hole 308. In one embodiment, the chamfered free end 147 of cantilever 142 engages the edge of middle phalanx 304 so as to cam cantilever 142 into biased relation with the bone, but without over stressing and damaging middle phalanx 304. Once fully inserted into middle phalanx 304, cantilever 142 provides additional resistance to removal of implant 125 from hole 308.

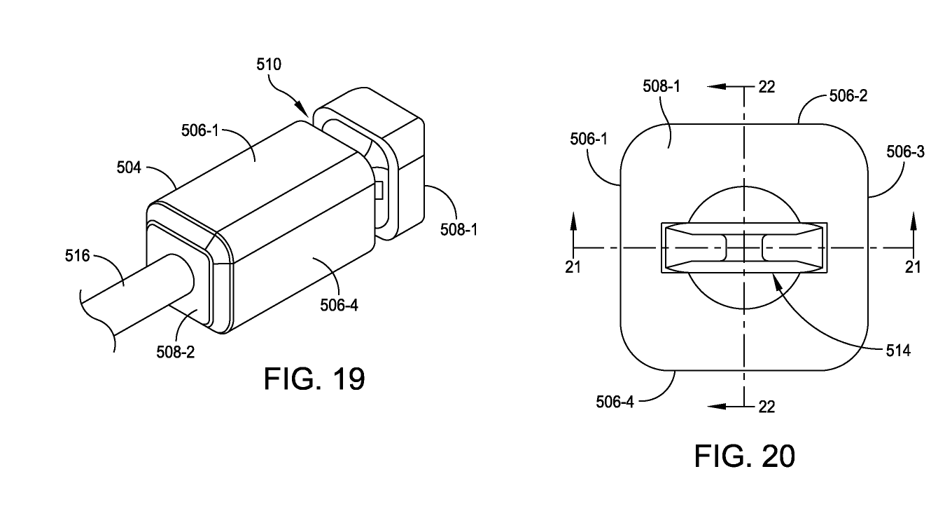

FIGS. 17-27 illustrate another embodiment of a driver assembly 500 for installing an implant into bone. As shown in FIGS. 17 and 18, driver assembly 500 includes an adapter 502 coupled to a driving rod 516 onto which a handle 534 is over-molded or otherwise coupled. Adapter 502 includes a body 504 with a substantially rectangular side profile comprising side walls 506-1, 506-2, 506-3, and 506-4 (collectively referred to as “side walls 506”) and a pair of end walls 508-1, 508-2 (collectively referred to as “end walls 508”) having a substantially square geometry as best seen in FIGS. 19-22.

Body 504 defines a recess 510 along the length of side walls 506. Recess 510 is dimensioned such that an o-ring 544 (FIGS. 17 and 18) may be received therein. Additionally, recess 510 is located along side walls 506 at a distance from end walls 508 such that recess 510 is aligned with a valley 126 of serrated edges 112 along the top and bottom sides 114, 116 of blade portion 104.

End wall 508-1 defines an aperture 512 having a geometry that complements the cross-sectional geometry of blade portion 104 of implant 100. For example, if implant 100 has a straight blade portion 104 as illustrated in FIG. 2, then aperture 512 may extend approximately parallel to the lengthwise direction of side walls 506. If the blade portion 104 of implant 100 is angled as illustrated in FIG. 6, then aperture 512 may extend from wall 508-1 at an angle relative to the plane defined by side wall 506-2 or 506-4 as will be understood by one skilled in the art. In some embodiments, aperture 512 has a depth that is greater than or equal to a length of blade portion 104 such that blade portion 104 may be received within body 504 and engagement portion 106 abuts end wall 508-1. Similarly, end wall 508-2 defines an aperture 514 that is sized and configured to receive an end of elongate driving rod 516 therein.

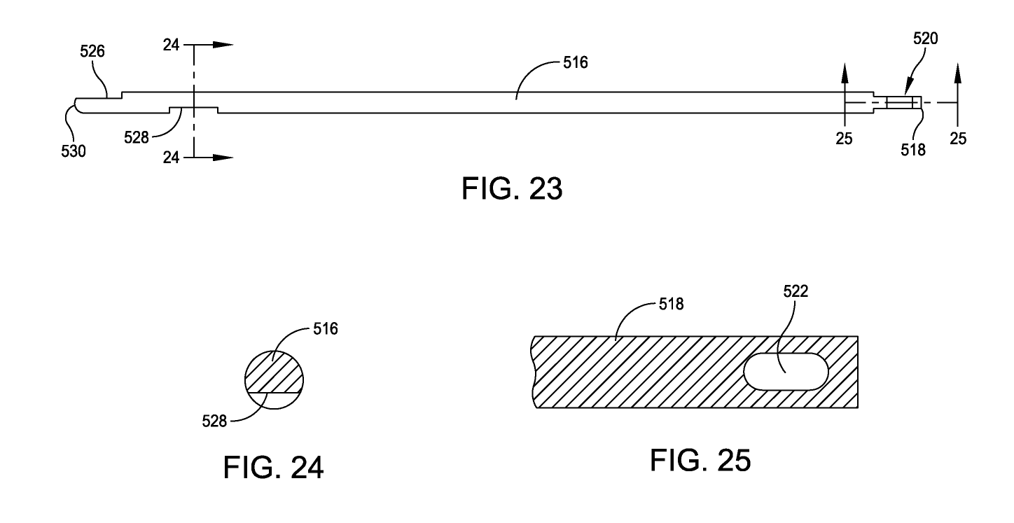

As best seen in FIGS. 23-25, driving rod 516 includes a fin 518 disposed at a first end 520. Fin 518 disposed at end 20 of driving rod 516 has a rectangular shape and is sized and configured to be received within aperture 512 of adapter 502. Fin 518 defines a slot 522, which is sized and configured to receive a pin (not shown) for cross-pinning driving rod 516 to adapter 502. In some embodiments, end 520 may have other cross-sectional geometries including, but not limited to, triangular, square, and pentagonal, to name a few possibilities, that are configured to be received within aperture 512. Adapter 502 may be over-molded onto the end of driving rod 516. However, one skilled in the art will understand that adapter 502 may be cross-pinned or otherwise coupled to driving rod 516.

The opposite end 524 of driving rod 516 defines a pair of flats 526, 528, which are disposed on opposite sides of driving rod 516. As best seen in FIG. 23, flat 526 extends from tip 530 and is linearly spaced from flat 528, which is disposed at a greater distance from tip 530 than flat 526. However, one skilled in the art will understand that flats 526, 528 may be disposed at other positions along driving rod 516. Flats 526, 528 are configured to provide a contact surface for coupling to handle 532, which may be over-molded onto driving rod 516, such that rotation of handle 532 is translated to driving rod 516.

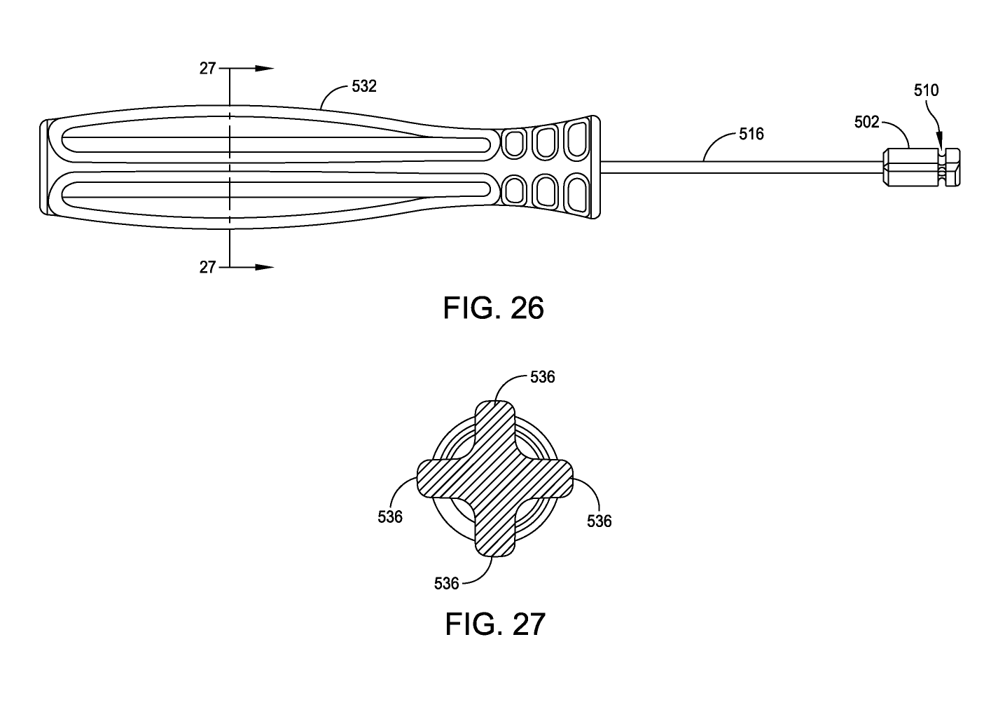

Turning now to FIGS. 26 and 27, handle 532 has an elongate body 534 that includes a plurality of ribs 536 that extend in a longitudinal direction along body 534 to provide a gripping surface for a user. As best seen in FIGS. 17 and 22, a smooth surface 538 interrupts circumferential ridges 540, which are disposed adjacent to proximal end 542 also for providing a gripping surface for a user.

Driver assembly 500 may be provided in a kit with a first adapter 502 for use with a straight implant 100 and a second adapter for use with an angled implant 100. A plurality of implants 100 of different sizes may also be provided in the kit. The kit may be used in an operation similar to the operation described above with respect to FIGS. 12A-16.

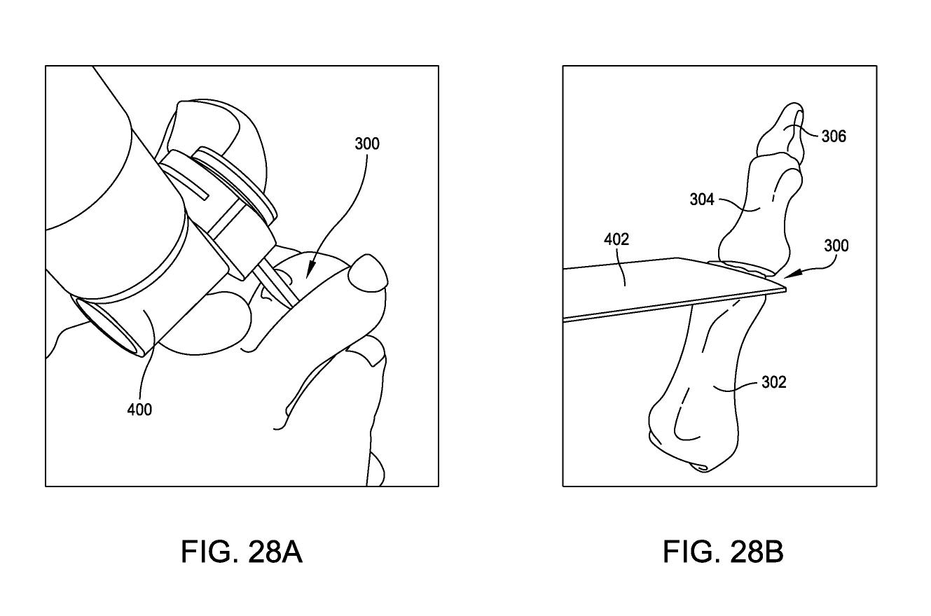

For example and referring to FIGS. 28A-33, an incision is made to open the PIP joint 300 and a cutting tool 400 having a blade 402 may be used to resect adjacent faces of proximal phalanx 302 and middle phalanx 304 as illustrated in FIGS. 28A and 28B. The resected surfaces of proximal phalanx 302 and middle phalanx 304 may be debrided as understood by one skilled in the art.

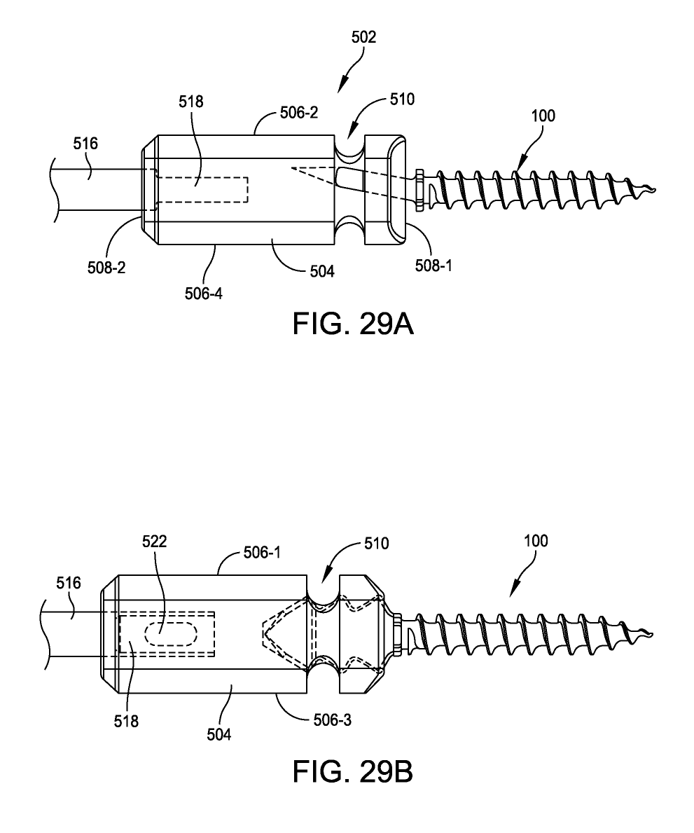

Blade portion 104 of implant 100 is disposed within aperture 512 of adapter 502 as shown in FIGS. 29A and 29B. With blade portion 104 disposed within aperture 512, an o-ring 544 (FIGS. 17 and 18) is placed in recess 510 defined by adapter 502 and within a valley 126 of serrated edges 112 along the top and bottom sides 114, 116 of blade portion 104. O-ring 544 secures implant 100 to adapter 502 such that implant does not move axially out of aperture 512.

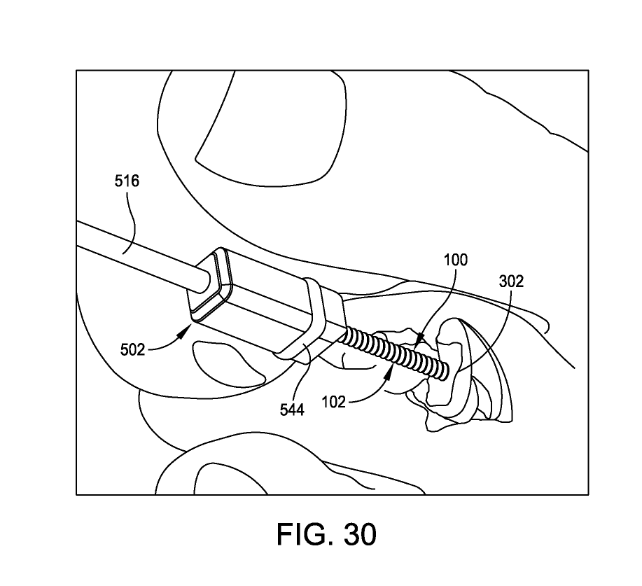

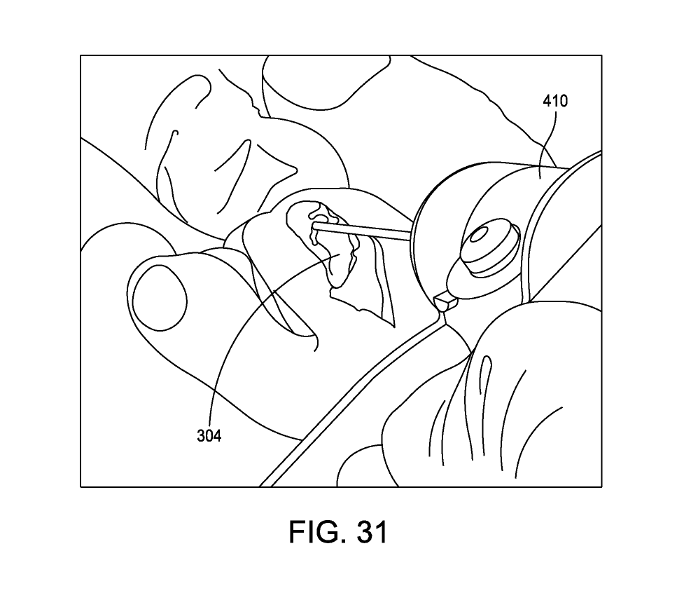

Once implant 100 is secured to adapter 502, the surgeon uses handle 534 to manually drive threaded portion 102 of implant 100 into the resected surface of proximal phalanx 302 as illustrated in FIG. 30. Implant 100 is driven into proximal phalanx 302 until engagement portion 106 abuts proximal phalanx 302. Implant 100 is decoupled from adapter 502 by axially pulling handle 534 away from implant 100 with sufficient force to flex o-ring 544 and separate adapter 502 from implant 100.

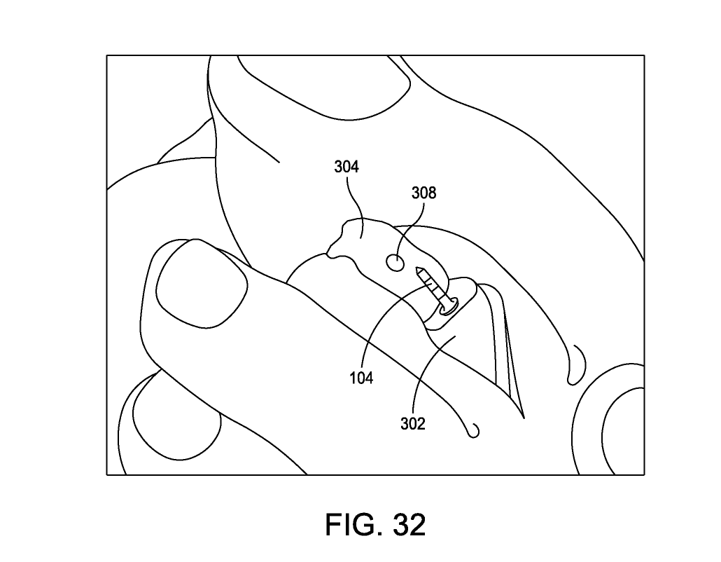

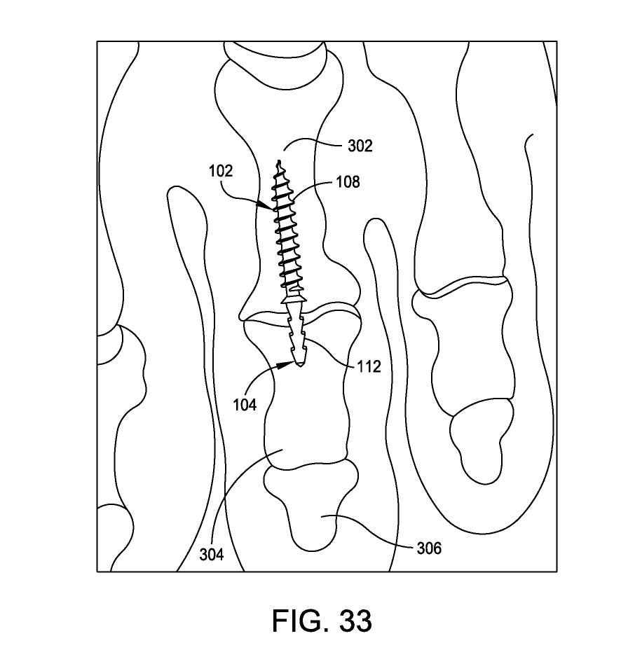

Middle phalanx 304 may be predrilled or broached using drill 410 to create a hole 308 as shown in FIGS. 31 and 32. The predrilled or broached middle phalanx 304 is then repositioned such that the predrilled hole or broach 308 aligns with the blade portion 104 of implant 100. The middle phalanx 304 is then pressed into engagement with the blade portion 104 as shown in FIG. 33. Serrated edges 112 of blade portion 104 help to maintain the engagement between middle phalanx 304 and blade portion 104 of implant 100.

The implant described above may advantageously be installed through a small incision as described above. Additionally, the improved implant is completely disposed within a toe of a patient, which prevents the implant from being caught on bed sheets or other objects like the conventional pins.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. An implant, comprising:
an elongate threaded portion; and
a blade extending from the elongate threaded portion, the blade having a top surface, a bottom surface, and a taper terminating at a point; and
a biasable cantilever formed in a central portion of said blade and having a free end that protrudes from at least one surface when said cantilever is unbiased.

2. The implant of claim 1, wherein the blade portion tapers along its width and thickness to the point.

3. The implant of claim 1, further comprising an engagement portion disposed between the threaded portion and the blade portion, the engagement portion including a projection extending in a direction away from an axis defined by the elongate threaded portion.

4. The implant of claim 3, wherein said free end of said cantilever is positioned adjacent to said engagement portion.

5. The implant of claim 3, wherein the direction is approximately perpendicular to the axis defined by the threaded portion.

6. The implant of claim 1, further comprising an engagement portion disposed between the threaded portion and the blade, the engagement portion including a pair of projections extending in opposite directions from an axis defined by an elongate threaded portion.

7. The implant of claim 6, wherein the blade extends from the elongate threaded member at an angle with respect to an axis defined by the elongate threaded member.

8. The implant of claim 7, wherein the angle is about 25 degrees.

9. The implant of claim 2, wherein said free end of said cantilever is positioned adjacent to said point.

10. The implant of claim 9, wherein said free end of said cantilever comprises a chamfered edge positioned proud of said blade.

11. A method, comprising:
forming an incision to gain access to a joint between first and second bones;
flexing the first and second bones such that the bones are disposed at an angle from one another;
advancing a threaded portion of an implant into the first bone, the implant including a blade extending from the elongate threaded portion wherein said blade includes a biasable cantilever having a free end that protrudes from at least one surface when said cantilever is unbiased;
repositioning the second bone such that a middle of the second bone is approximately aligned with the blade of the implant; and
forcing the second bone into engagement with the blade portion of the implant and therbu biasing said cantilever.

12. The method of claim 11, further comprising:
at least one of pre-drilling and broaching the second bone prior to forcing the second bone into engagement with the blade portion of the implant such that said biased cantilever engages said bone.

13. The method of claim 11, further comprising:
resecting an end of each the first and second bones prior to advancing the elongate threaded portion of the implant into the first bone.

14. The method of claim 11, wherein the first bone is one of a proximal phalanx or a middle phalanx of a foot, and the second bone is the other of the proximal phalanx or the middle phalanx of a foot.

15. The method of claim 11, wherein the blade of the implant extends from the elongate threaded member at an angle with respect to an axis defined by the elongate threaded member.

16. The method of claim 11, wherein the implant includes an engagement portion disposed between the threaded portion and the blade portion, the engagement portion including a projection extending from an axis defined by the elongate threaded portion.

17. A surgical assembly, comprising:
an implant including:
a threaded ended, and
a blade end extending from the threaded end, the blade end tapering along its thickness and its width to a point and including a plurality of serrated edges and a biasable cantilever formed in a central portion of said blade and having a free end that protrudes from at least one surface when said cantilever is unbiased; and
a driving assembly including:
a handle,
a driving rod extending from the handle, and
an adapter coupled to an end of the driving rod, the adapter having a body defining a slot at one end that is sized and configured to receive the blade end of the implant.

18. The surgical assembly of claim 17, wherein the blade end extends from the threaded end in a direction that is disposed at an angle with respect to an axis defined by the threaded end.

19. The surgical assembly of claim 18, wherein the adapter body defines a circumferential groove sized and configured to receive an o-ring for coupling the implant to the adapter.

20. The surgical assembly of claim 17, wherein the o-ring is disposed within a valley of at least one of the serrated edges of the implant when the implant is disposed within the slot defined by the adapter.

21. The surgical assembly of claim 17, wherein the implant includes an engagement portion disposed between the threaded portion and the blade, the engagement portion including a projection extending in a direction away from an axis defined by the elongate threaded portion.

22. The surgical assembly of claim 21, wherein the engagement portion includes a pair of projections radially extending in opposite directions from one another.
